(19) United States
(12) Patent Application Publication
(10) Pub. No.: US 2016/0157718 A1
(43) Pub. Date: Jun. 9, 2016

(54) CHOOSING A HEART RATE MONITOR FOR A WEARABLE MONITORING DEVICE

(71) Applicant: WiseWear Corporation, San Antonio, TX (US)

(72) Inventors: RONALD A. BARNES, SAN ANTONIO, TX (US); JASON A. BEENS, HELOTES, TX (US); DAVID P. ELAM, JR., SAN ANTONIO, TX (US); BENNETT L. IBEY, SAN ANTONIO, TX (US); GERALD J. WILMINK, SAN ANTONIO, TX (US)

(21) Appl. No.: 14/564,168

(22) Filed: Dec. 9, 2014

(51) Int. Cl. A61B 5/00; A61B 5/0205; A61B 5/11; A61B 5/0402

(52) U.S. Cl. CPC A61B 5/0024; A61B 5/0402; A61B 5/6823; A61B 5/6824; A61B 5/0205; A61B 5/1118; A61B 5/486; A61B 5/684; A61B 5/6898; A61B 5/0006; A61B 2560/0456; A61B 5/02438

(57) ABSTRACT

Methods, apparatuses, and computer readable mediums for choosing a heart rate monitor for a wearable monitoring device. In a particular embodiment, the wearable monitoring device identifies a location of a wearable monitoring device on a user; selects based on an identification of the location, a heart rate monitor from the plurality of heart rate monitors; and uses the selected heart rate monitor to monitor a heart rate of the user.

### BACKGROUND

1. Field of the Invention

The field of the invention is data processing, or, more specifically, methods, apparatuses, and computer readable mediums for choosing a heart rate monitor for a wearable monitoring device.

2. Description of Related Art

The health benefits of regular exercise and physical activity are well documented. To aid in the encouragement and evaluation of a performance of physical activity, people are using wearable monitoring devices with multiple sensors. However, in a device with multiple sensors, determining which sensor to use to monitor an activity of the user may be difficult.

### SUMMARY

Methods, apparatuses, and computer readable mediums for choosing a heart rate monitor for a wearable monitoring device. In a particular embodiment, the wearable monitoring device identifies a location of a wearable monitoring device on a user; selects based on an identification of the location, a heart rate monitor from the plurality of heart rate monitors; and uses the selected heart rate monitor to monitor a heart rate of the user.

In another embodiment, the wearable monitoring device generates an activity level measurement based on sensed motion of the user; based on the activity level measurement, generates a recommendation indicating a recommended location to place a wearable monitoring device on the user; and provides the recommendation to the user.

The foregoing and other objects, features and advantages of the present disclosure will become apparent after review of the entire application, including the following sections: Brief Description of the Drawings, Detailed Description, and the Claims.

### BRIEF DESCRIPTION OF THE DRAWINGS

FIG. 1 sets forth a diagram of an illustrative embodiment of an apparatus for choosing a heart rate monitor for a wearable monitoring device.

FIG. 2 sets forth a block diagram of another illustrative embodiment of an apparatus for choosing a heart rate monitor for a wearable monitoring device.

FIG. 3 sets forth a flow chart illustrating an example embodiment of a method for choosing a heart rate monitor for a wearable monitoring device.

FIG. 4 sets forth a flow chart illustrating another example embodiment of a method for choosing a heart rate monitor for a wearable monitoring device.

FIG. 5 sets forth a flow chart illustrating another example embodiment of a method for choosing a heart rate monitor for a wearable monitoring device.

FIG. 6 sets forth a flow chart illustrating another example embodiment of a method for choosing a heart rate monitor for a wearable monitoring device.

FIG. 7 sets forth a flow chart illustrating another example embodiment of a method for choosing a heart rate monitor for a wearable monitoring device.

FIG. 8 sets forth a flow chart illustrating another example embodiment of a method for choosing a heart rate monitor for a wearable monitoring device.

FIG. 9 sets forth a data flow diagram illustrating a manufacturing process of a device configured for choosing a heart rate monitor for a wearable monitoring device.

### DETAILED DESCRIPTION

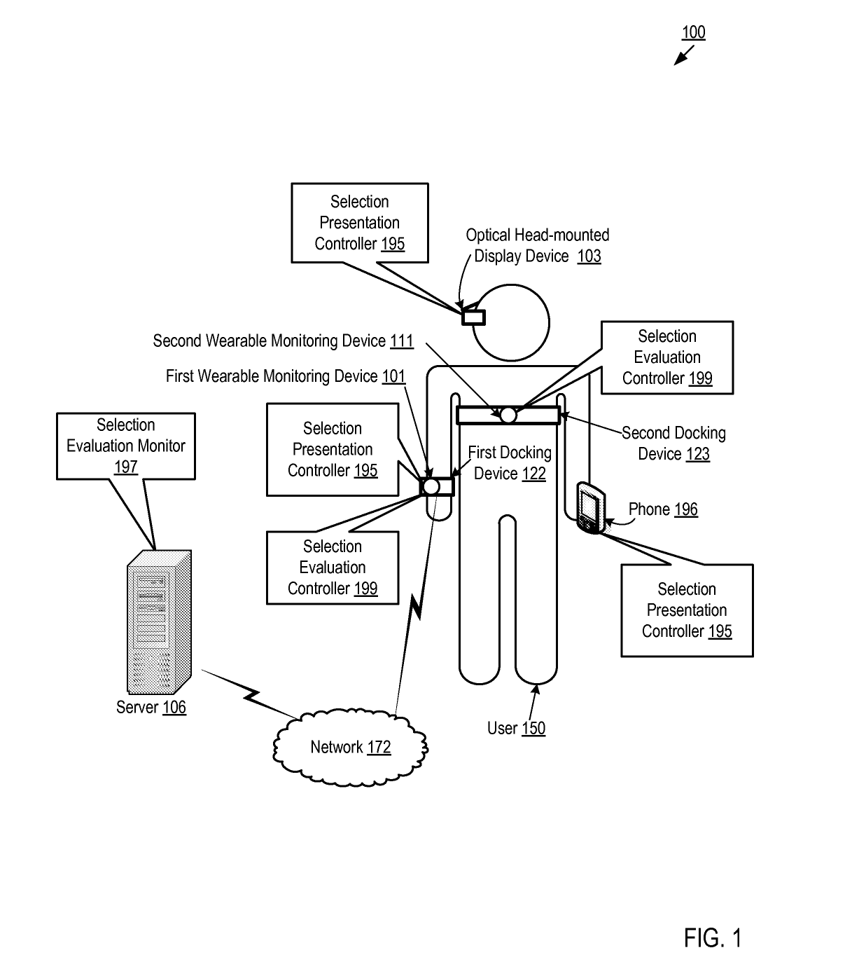

FIG. 1 sets forth a diagram of an illustrative embodiment of an apparatus (100) for choosing a heart rate monitor for a wearable monitoring device (150). In the example of FIG. 1, the apparatus (100) includes a first wearable monitoring device (101), a second wearable monitoring device (111), an optical head-mounted display device (103), a phone (196), and a server (106).

A wearable monitoring device is any device that is wearable and includes automated computing machinery for monitoring a user. Non-limiting examples of wearable monitoring devices include smart wristwatches, bracelets, straps, pendants, and any other forms of wearable devices capable of monitoring a user. In the example of FIG. 1, the first wearable monitoring device (101) is coupled to a first docking device (122) for connecting the first wearable monitoring device (101) to the wrist of the user (150) in a wrist monitoring location. The second wearable monitoring device is coupled to a second docking device (123) for connecting the second wearable monitoring device (11) to the chest of the user (150) in a chest monitoring location. Readers of skill in the art will recognize that wearable monitoring devices may be placed on any number of locations on a user.

As part of ‘monitoring’ a user, a wearable monitoring device may be configured to utilize data from one or more sensors that are either coupled to the wearable monitoring device or are part of the wearable monitoring device. Non-limiting examples of the types of sensors that may be available to a wearable monitoring device include a hydration sensor, an optical heart rate monitor, an ECG monitor, a thermometer, an electromyography (EMG) sensor, an accelerometer, a gyroscope, a Global Positioning System (GPS) location sensor, an environmental condition sensor, and many other types of sensors that would be useful for monitoring a user.

To acquire data from these sensors, a wearable monitoring device may include data acquisition (DAQ) hardware for periodically polling or receiving data from one or more of the sensors available to the wearable monitoring device. For example, circuitry within the wearable monitoring device may monitor the existence and strength of a signal from a sensor and process any signals received from the sensor. The wearable monitoring device (101, 111) may also include circuitry for processing the sensor data. For example, the wearable monitoring device (101, 111) may include circuitry for converting sensor data to another data form, such as physiological data and environmental condition data. That is, the wearable monitoring device (101, 111) of FIG. 1 may include the computing components necessary to receive, process, and transform sensor data into a type of data that is usable in a process for choosing a heart rate monitor for a wearable monitoring device.

In the example of FIG. 1, the wearable monitoring device (101, 111) includes a selection evaluation controller (199) comprised of automated computing machinery configured for choosing a heart rate monitor. As part of the process of choosing a heart rate monitor for a wearable monitoring device, the selection evaluation controller (199) may be configured to identify a location of the wearable monitoring device (101, 111) on a user (150). A wearable monitoring device may be capable of monitoring a user from a variety of locations on a user. For example, a wearable monitoring device may be indirectly coupled to the user, such as in the user's pocket or attached to the user's shoe. In other examples, a wearable monitoring device may be directly coupled to the user's body, such as to the user's wrist or the user's chest. The location of the wearable monitoring device on the user may directly impact the operation of the sensors of the wearable monitoring device. For example, a ECG heart rate monitor may not be able to detect an ECG signal from a user's heart unless the wearable monitoring device that includes the ECG heart rate monitor is in a chest monitoring location on the user. As such, identifying the location of the wearable monitoring device on the user may be useful for choosing a heart rate monitor for a wearable monitoring device.

Identifying a location of the wearable monitoring device (101, 111) on the user (150) may be carried out by receiving user input, which directly or indirectly indicates the location of the wearable monitoring device. For example, the user (150) may use an interface of the wearable monitoring device (150) or other user device to indicate the location through the act of depressing a button, speaking, or writing.

In a particular embodiment, identifying a location of the wearable monitoring device (101, 111) on the user (150) may be carried out by analyzing sensor input to identify the location of the wearable monitoring device. For example, the selection evaluation controller (199) may determine that output from one or more sensors corresponds to the wearable monitoring device (101, 111) being in a particular location on the user (150).

As part of the process for choosing a heart rate monitor for a wearable monitoring device, the selection evaluation controller (199) may also be configured to select, based on the identified location, a heart rate monitor from a plurality of heart rate monitors. Selecting, based on the identified location, a heart rate monitor from a plurality of heart rate monitors may be carried out by examining a table containing entries that match a particular location with one or more heart rate monitors.

The selection evaluation controller (199) may also be configured to use the selected heart rate monitor to monitor a heart rate of the user (150). Using the selected heart rate monitor to monitor a heart rate of the user may be carried out by transmitting a selection signal to the selected heart rate monitor; periodically polling or querying the selected heart rate monitor to retrieve sensor data from the selected heart rate monitor; and receiving sensor data from the selected heart rate monitor.

Choosing a heart rate monitor for a wearable monitoring device may also include the selection evaluation controller (199) being configured to generate an activity level measurement based on sensed motion of the user (150). The sensed motion of the user may be indicated by motion data generated by one or more motion sensors of the wearable monitoring device. Non-limiting examples of motion data include acceleration data and gyroscope data, generated by an accelerometer and a gyroscope, respectively. Generating an activity level measurement based on sensed motion of the user (150) may be carried out by determining an activity level measurement that indicates an intensity level of the activity of the user. For example, an activity level measurement may indicate an absolute value of acceleration data indicating changes in the direction of motion by the user.

As part of the process of choosing a heart rate monitor for a wearable monitoring device, the selection evaluation controller (199) may be configured to generate, based on the activity level measurement, a recommendation indicating a recommended location to place the wearable monitoring device on the user (150). Generating a recommendation indicating a recommended location to place the wearable monitoring device on the user (150) may be carried out by determining which heart rate sensor best corresponds to the intensity level of the user's exercise activity; and storing an indication of the recommended location within a visual output message or an audio output message. For example, a selection evaluation controller may determine that an ECG heart rate monitor is more accurate at higher levels of exercise activity than an optical heart rate monitor. In this example, the selection evaluation monitor (199) may recommend that a user move the wearable monitoring device to the chest monitoring location so that the ECG heart rate monitor may be used.

The selection evaluation controller may also be configured to provide the recommendation to the user (150). Providing the recommendation to the user (150) may be carried out by sending a recommendation message to the user suggesting a position for the wearable monitoring device. In a particular embodiment, the recommendation message is a visual output message or an audio output message.

As another example, the selection evaluation controller (199) may transmit a visual output to a screen of the wearable monitoring device (101). In this example, the visual output may display the recommendation. Examples of visual output may include a displayed message, an animated avatar showing the recommended location, and many others as will occur to Readers of skill in the art.

In a particular embodiment, providing the recommendation may include transmitting the recommendation to a selection presentation controller (195). A selection presentation controller includes automated computing machinery configured to receive the recommendation and to present the recommended location within the recommendation to a user. In the example of FIG. 1, the phone (196) and the optical head-mounted display device (103) both include the selection presentation controller (195). The phone (196) and the optical head-mounted display device (103) may include visual and audio outputs for providing the recommendation to the user (150). The first wearable monitoring device (101) of FIG. 1 also includes the selection presentation controller (195). However, Readers of skill in the art will realize that the selection presentation controller (195) and the selection evaluation controller (199) need not be incorporated into the same device, as shown in the second wearable monitoring device (111), which includes the selection evaluation controller (199) but not the selection presentation controller (195).

The selection evaluation controller (199) may also be configured to provide the recommendation to another device. For example, the selection evaluation controller (199) may provide the indication of the recommendation to a server (106) via a network (172). The server (106) includes a selection evaluation monitor (197). The selection evaluation monitor (197) may include automated computing machinery configured to receive the recommendation. The recommendation evaluation monitor (197) may also be configured to act as a database repository for that stores physiological data, environmental condition data, and any other type of data that the selection evaluation controller (199) may utilize to generate and provide the recommendation to the user. The selection evaluation monitor (197) may be configured to provide this stored data to the selection evaluation controller (199).

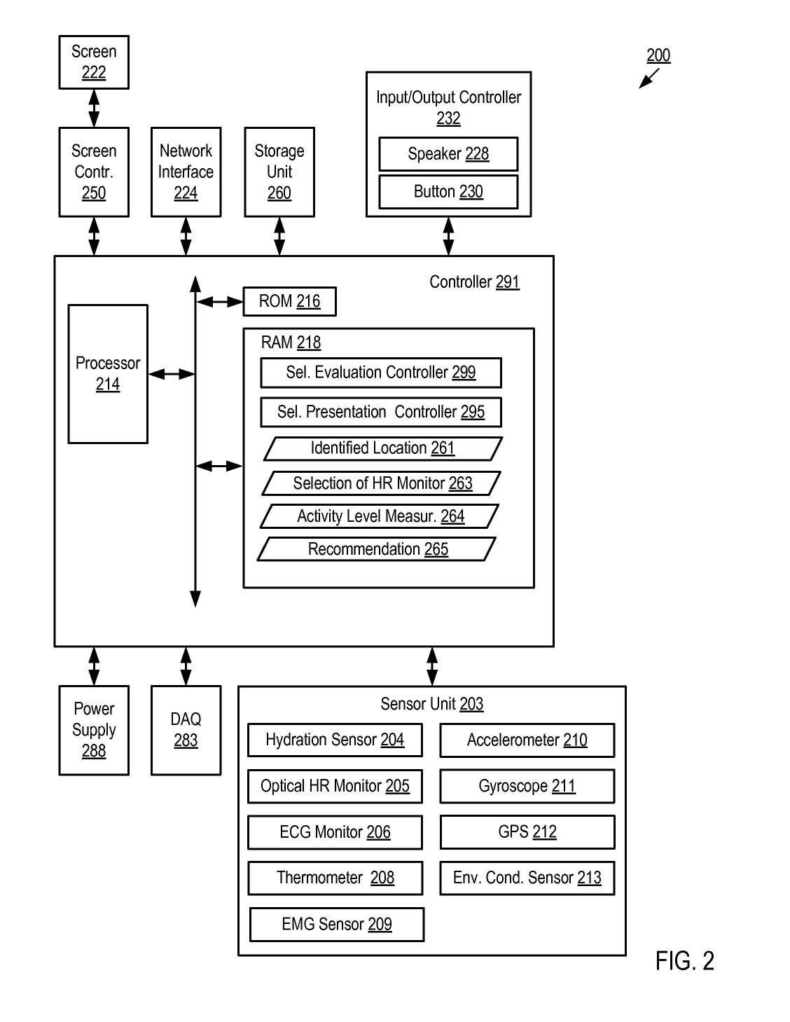

Referring to FIG. 2, an illustrative apparatus (200) for choosing a heart rate monitor for a wearable monitoring device is shown. The apparatus (200) is an example wearable monitoring device that includes a controller (291) that includes a processor (214), read only memory (ROM) (216), random access memory (RAM) (218). The RAM (218) includes a selection evaluation controller (299) and a selection presentation controller (295). Although, in the example of FIG. 2, the selection evaluation controller (299) and the selection presentation controller (295) are included in RAM (218), Readers of skill in the art will recognize that the selection evaluation controller (299) and the selection presentation controller (295) may be included in other storage locations, such as the ROM (216) and an external storage unit (260), which is coupled for data communications with the controller (291).

In the example of FIG. 2, the apparatus (200) also includes a power supply (288), a screen (222), a screen controller (250), a network interface (224), an input/output controller (232) having a speaker (228) and a button (230), a data acquisition processing unit (DAQ) (283), and a sensor unit (203). The sensor unit (203) of FIG. 2 includes sensors for generating, capturing, and transmitting motion data. In the example of FIG. 2, the sensor unit (203) includes an accelerometer (210), a gyroscope (211), and a global positioning system unit (212).

An accelerometer measures proper acceleration, which is the acceleration it experiences relative to freefall and is the acceleration felt by people and objects. Put another way, at any point in spacetime the equivalence principle guarantees the existence of a local inertial frame, and an accelerometer measures the acceleration relative to that frame. Such accelerations are popularly measured in terms of g-force. Conceptually, an accelerometer behaves as a damped mass on a spring. When the accelerometer experiences an acceleration, the mass is displaced to the point that the spring is able to accelerate the mass at the same rate as the casing. The displacement is then measured to give the acceleration. Modern accelerometers are often small micro electro-mechanical systems (MEMS), and are indeed the simplest MEMS devices possible, consisting of little more than a cantilever beam with a proof mass (also known as seismic mass). Damping results from the residual gas sealed in the device. As long as the Q-factor is not too low, damping does not result in a lower sensitivity. Most micromechanical accelerometers operate in-plane, that is, they are designed to be sensitive only to a direction in the plane of the die. By integrating two devices perpendicularly on a single die a two-axis accelerometer can be made. By adding another out-of-plane device three axes can be measured. Such a combination may have much lower misalignment error than three discrete models combined after packaging. Micromechanical accelerometers are available in a wide variety of measuring ranges, reaching up to thousands of g's.

A gyroscope is a device for measuring or maintaining orientation, based on the principles of angular momentum. Mechanical gyroscopes typically comprise a spinning wheel or disc in which the axle is free to assume any orientation. Although the orientation of the spin axis changes in response to an external torque, the amount of change and the direction of the change is less and in a different direction than it would be if the disk were not spinning. When mounted in a gimbal (which minimizes external torque), the orientation of the spin axis remains nearly fixed, regardless of the mounting platform's motion. Gyroscopes based on other operating principles also exist, such as the electronic, microchip-packaged MEMS gyroscope devices found in consumer electronic devices, solid-state ring lasers, fibre optic gyroscopes, and the extremely sensitive quantum gyroscope. A MEMS gyroscope takes the idea of the Foucault pendulum and uses a vibrating element, known as a MEMS (Micro Electro-Mechanical System). The integration of the gyroscope has allowed for more accurate recognition of movement within a 3D space than the previous lone accelerometer within a number of smartphones. Gyroscopes in consumer electronics are frequently combined with accelerometers (acceleration sensors) for more robust direction- and motion-sensing.

The Global Positioning System (GPS) is a space-based satellite navigation system that provides location and time information in all weather conditions, anywhere on or near the Earth where there is an unobstructed line of sight to four or more GPS satellites. In general, GPS receivers are composed of an antenna, tuned to the frequencies transmitted by the satellites, receiver-processors, and a highly stable clock (often a crystal oscillator).

The sensor unit (203) of FIG. 2 also includes a sensor (213) for generating, capturing, and transmitting environmental condition data. Environmental condition data may include any indications of the environment of the user. In a particular embodiment, environmental condition data may indicate weather conditions, such as humidity level, precipitation measurements, cloud coverage, and temperature. Environmental condition data may also indicate whether the user is inside or outside. For example, a user may provide input to the wearable monitoring device indicating that the user is indoors. In another embodiment, environmental condition data may be measured by the wearable monitoring device. For example, the wearable monitoring device may include a sensor that monitors humidity level or temperature surrounding the wearable monitoring device. In another embodiment, the wearable monitoring device may use one or more network interfaces to receive indications of environmental conditions, such as from a weather indication application, or from a local environmental condition indication device, such as a networked humidity and temperature sensor.

The sensor unit (203) of FIG. 2 also includes sensors for generating, capturing, and transmitting physiological data. In the example of FIG. 2, the sensor unit (203) includes a hydration sensor (204), an optical heart rate monitor (205), an electrocardiograph (ECG) monitor (206), a thermometer (208), and an electromyograph (209) for performing electromyography (EMG).

A hydration sensor may be any type of sensor capable of measuring a hydration level of a person. Measuring a hydration level of a person may be performed by a variety of methods via a variety of systems, including but not limited to measuring transepidermal water loss (TWL) with a skin hydration probe. TWL is defined as the measurement of the quantity of water that passes from inside a body through the epidermal layer (skin) to the surrounding atmosphere via diffusion and evaporation processes.

An ECG monitor also generates an activity pattern based on electrical activity of the heart. On the ECG, instantaneous heart rate is typically calculated using the R wave-to-t wave (RR) interval and multiplying/dividing in order to derive heart rate in heartbeats/min.

An optical heart rate monitor may be part of a pulse oximeter. A pulse oximeter is a medical device that indirectly monitors the oxygen saturation of a user's blood (as opposed to measuring oxygen saturation directly through a blood sample) and changes in blood volume in the skin, producing a photoplethysmogram. A typical pulse oximeter utilizes an electronic processor and a pair of small light-emitting diodes (LEDs) facing a photodiode through a translucent part of the patient's body, usually a fingertip or an earlobe. Typically one LED is red, with wavelength of 660 nm, and the other is infrared with a wavelength of 940 nm. Absorption of light at these wavelengths differs significantly between blood loaded with oxygen and blood lacking oxygen. Oxygenated hemoglobin absorbs more infrared light and allows more red light to pass through. Deoxygenated hemoglobin allows more infrared light to pass through and absorbs more red light. The LEDs flash about thirty times per second which allows the photodiode to respond to the red and infrared light separately. The amount of light that is transmitted (in other words, that is not absorbed) is measured, and separate normalized signals are produced for each wavelength. These signals fluctuate in time because the amount of arterial blood that is present increases (literally pulses) with each heartbeat. By subtracting the minimum transmitted light from the peak transmitted light in each wavelength, the effects of other tissues is corrected for. The ratio of the red light measurement to the infrared light measurement is then calculated by the processor (which represents the ratio of oxygenated hemoglobin to deoxygenated hemoglobin), and this ratio is then converted to SpO2 by the processor via a lookup table.

A thermometer is a device that measures temperature or a temperature gradient. A thermistor is an example of a type of thermometer that may be used to measure temperature. A thermistor is a type of resistor whose resistance varies significantly with temperature, more so than in standard resistors. Thermistors are widely used as inrush current limiters, temperature sensors, self-resetting overcurrent protectors, and self-regulating heating elements.

An electromyograph detects the electrical potential generated by muscle cells when these cells are electrically or neurologically activated. The signals can be analyzed to detect medical abnormalities, activation level, or recruitment order or to analyze the biomechanics of human or animal movement.

The data acquisition (DAQ) hardware (283) is configured for periodically polling or receiving data from one or more sensors. For example, circuitry within the DAQ (283) may monitor the existence and strength of a signal from a sensor and process any signals received the sensor. The DAQ (283) may also include circuitry for processing the sensor data. For example, the DAQ (283) may include circuitry for conversion of sensor data to another data form, such as motion data, physiological data, or environmental condition data. That is, the DAQ (283) of FIG. 2 may include the computing components necessary to receive, process, and transform sensor data into a type of data that is usable in a process for choosing a heart rate monitor for a wearable monitoring device.

The selection evaluation controller (299) comprises automated computing machinery configured for choosing a heart rate monitor for a wearable monitoring device of a user. Specifically, the selection evaluation controller (299) may be configured to identifying a location of a wearable monitoring device (200) on a user; select based on an identification (261) of the location, a heart rate monitor from the plurality of heart rate monitors (205, 206); and use the selected heart rate monitor to monitor a heart rate of the user. In the example of FIG. 2, selecting a heart rate monitor may include generating a selection (263) representing the selected heart rate monitor.

In a particular embodiment, the selection evaluation controller (299) may be configured to generate, an activity level measurement (264) based on sensed motion of the user; generate based on the activity level measurement (264), a recommendation (265) indicating a recommended location to place a wearable monitoring device on the user; and provide the recommendation (265) to the user.

The selection presentation controller (295) comprises automated computing machinery configured for choosing a heart rate monitor for a wearable monitoring device of a user. Specifically, the selection presentation controller (295) is configured to receive the recommendation and to present the recommendation to the user. In the example of FIG. 2, the selection presentation controller (295) may provide the recommendation by: generating and transmitting visual output that is used to display a message within a graphical user interface displayed on the screen (222); and generating and transmitting audio output that is used to play audio over on the speaker (228).

The controller (291) is also coupled to a network interface (224), such as an Ethernet port, modem port or other network port adapter. The network interface (224) is adapted to connect to a network and to send data to a selection presentation controller or a selection evaluation monitor located on a separate device. The network may include one or a combination of any type of network such as LAN, WAN, WLAN, public switched telephone network, GSM, or otherwise.

In a particular embodiment, the power supply (288) may include circuitry used for inductive charging. Inductive charging (also known as “wireless charging”) uses an electromagnetic field to transfer energy between two objects. This is usually done with a charging station. Energy is sent through an inductive coupling to an electrical device, which can then use that energy to charge batteries or run the device. Induction chargers typically use an induction coil to create an alternating electromagnetic field from within a charging base station, and a second induction coil in the portable device takes power from the electromagnetic field and converts it back into electrical current to charge the battery. The two induction coils in proximity combine to form an electrical transformer. Greater distances between sender and receiver coils can be achieved when the inductive charging system uses resonant inductive coupling. Recent improvements to this resonant system include using a movable transmission coil (i.e., mounted on an elevating platform or arm), and the use of advanced materials for the receiver coil made of silver plated copper or sometimes aluminum to minimize weight and decrease resistance due to the skin effect.

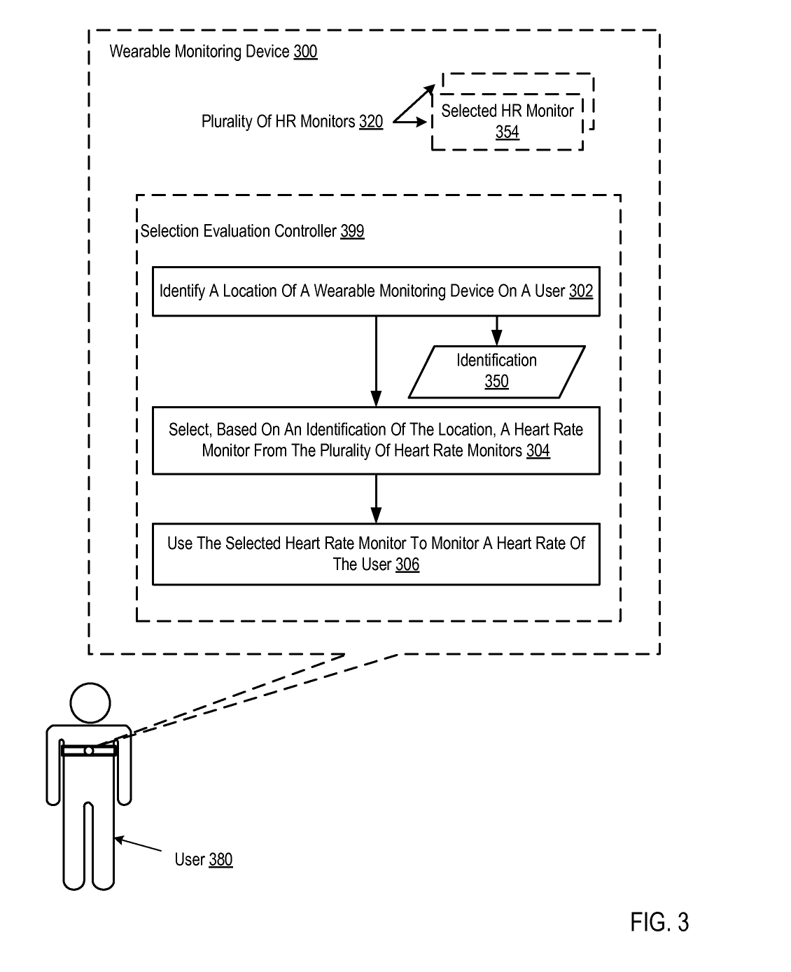

For further explanation, FIG. 3 sets forth a flow chart illustrating an example embodiment of a method for choosing a heart rate monitor for a wearable monitoring device. As part of the process for choosing a heart rate monitor for a wearable monitoring device, the method of FIG. 3 includes a selection evaluation controller (399) of a wearable monitoring device (300) identifying (302) an identification (350) of the location of the wearable monitoring device (300) on a user (380). A wearable monitoring device may be capable of monitoring a user from a variety of locations on a user. For example, a wearable monitoring device may be indirectly coupled to the user, such as in the user's pocket or attached to the user's shoe. In other examples, a wearable monitoring device may be directly coupled to the user's body, such as to the user's wrist or the user's chest. The location of the wearable monitoring device on the user may directly impact the operation of the sensors of the wearable monitoring device. For example, a ECG heart rate monitor may not be able to detect an ECG signal from a user's heart unless the wearable monitoring device that includes the ECG heart rate monitor is in a chest monitoring location on the user. As such, identifying the location of the wearable monitoring device on the user may be useful for choosing a heart rate monitor for a wearable monitoring device.

Identifying (302) a location of the wearable monitoring device (300) on a user (380) may be carried out by receiving user input directly or indirectly indicating the location of the wearable monitoring device. For example, the user (380) may use an interface of the wearable monitoring device (300) or other user device to indicate the location through the act of depressing a button, speaking, or writing.

Identifying (302) a location of the wearable monitoring device (300) on a user (380) may be carried out by analyzing sensor input to identify the location of the wearable monitoring device. For example, the selection evaluation controller (399) may determine that output from one or more sensors corresponds to the wearable monitoring device (300) being in a particular location on the user (380).

As part of the process for choosing a heart rate monitor for a wearable monitoring device, the method of FIG. 3 also includes the selection evaluation controller (399) selecting (304), based on an identification (350) of the location, a heart rate monitor (354) from a plurality of heart rate monitors (320). Selecting (304), based on an identification (350) of the location, a heart rate monitor (354) from a plurality of heart rate monitors (320) may be carried out by examining a table containing entries that match a particular location with one or more heart rate monitors.

The method of FIG. 3 also includes the selection evaluation controller (399) using (306) the selected heart rate monitor (354) to monitor a heart rate of the user (380). Using (306) the selected heart rate monitor (354) to monitor a heart rate of the user (380) may be carried out by transmitting a selection signal to the selected heart rate monitor; periodically polling or querying the selected heart rate monitor to retrieve sensor data from the selected heart rate monitor; and receiving sensor data from the selected heart rate monitor.

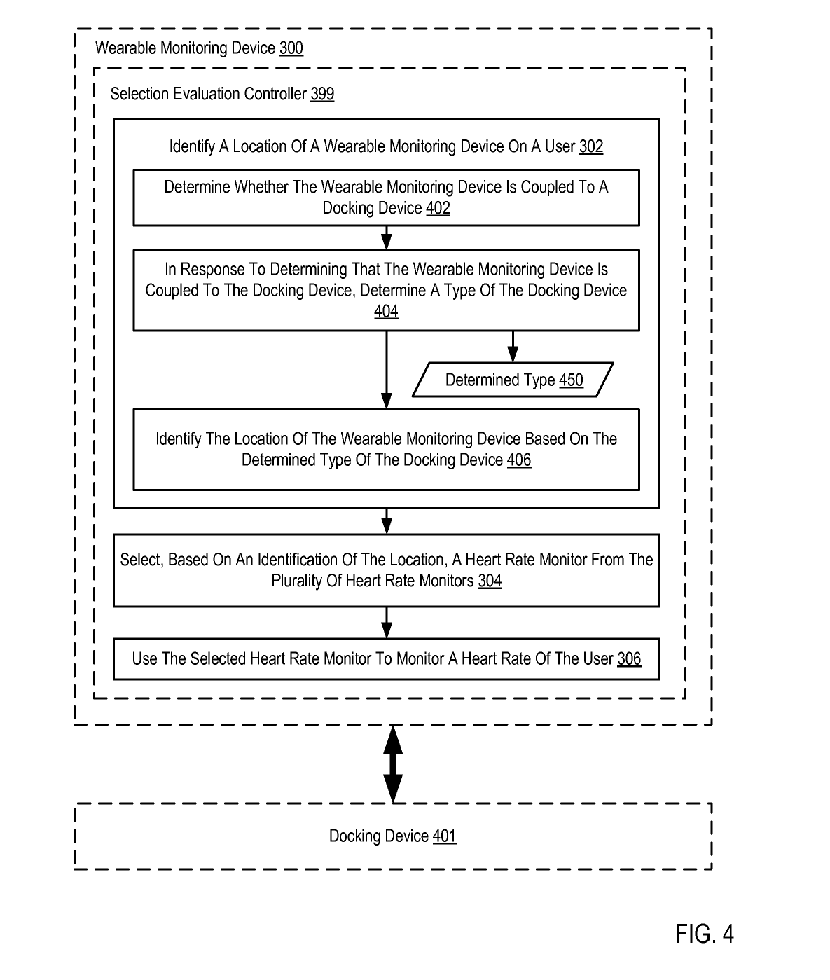

For further explanation, FIG. 4 sets forth a flow chart illustrating another example embodiment of a method for choosing a heart rate monitor for a wearable monitoring device. The method of FIG. 4 is similar to the method of FIG. 3 in that the method of FIG. 4 also includes a selection evaluation controller (399) identifying (302) a location of the wearable monitoring device (300) on a user (380); selecting (304), based on an identification (350) of the location, a heart rate monitor (354) from a plurality of heart rate monitors (320); and using (306) the selected heart rate monitor (354) to monitor a heart rate of the user (380).

In the method of FIG. 4, however, identifying (302) a location of the wearable monitoring device (300) on a user (380) includes determining (402) whether the wearable monitoring device (300) is coupled to a docking device (401). A docking device is any type of device that couples to the wearable monitoring device. Non-limiting examples of docking devices include a chest strap docking device and a wrist strap docking device. Determining (402) whether the wearable monitoring device (400) is coupled to a docking device (401) may be carried out by receiving sensor input that indicates the wearable monitoring device is coupled to a docking device. For example, the wearable monitoring device may include an interface for coupling to the docking device. In this example, the interface may generate an electrical signal that indicates when the wearable monitoring device is coupled to a docking device.

In the example of FIG. 4, identifying (302) a location of the wearable monitoring device (300) on a user (380) also includes determining (404) a type (450) of the docking device (401) in response to determining that the wearable monitoring device (300 is coupled to the docking device (401). A docking device may be associated with a specific location on the user. A type of the docking device may therefore provide information that can identify the location of the wearable monitoring device. For example, a chest strap docking device may be a specific type of device for attaching the wearable monitoring device to the chest of the user. In this example, if the type of the docking device is identified as a chest strap docking device, the selection evaluation controller (399) may identify the location of the wearable monitoring device as a chest monitoring location on the user.

Determining (404) a type (450) of the docking device (401) in response to determining that the wearable monitoring device (300 is coupled to the docking device (401) may be carried out by receiving an electrical code from the interface between the wearable monitoring device and the docking device; receiving from the docking device, a wireless signal indicating the type of the docking device; and receiving user input indicating the type of the docking device. For example, a first set of pins in the interface may be engaged when a first type of docking device is coupled to the wearable monitoring device whereas a second set of pins in the interface may be engaged when a second type of docking device is coupled to the wearable monitoring device.

In the example of FIG. 4, identifying (302) a location of the wearable monitoring device (300) on a user (380) also includes identifying (406) the location of the wearable monitoring device (300) based on the determined type of the docking device (401). Identifying (406) the location of the wearable monitoring device (300) based on the determined type of the docking device (401) may be carried out by examining a table containing entries that match a particular type of a docking device with a location of the wearable monitoring device on the user.

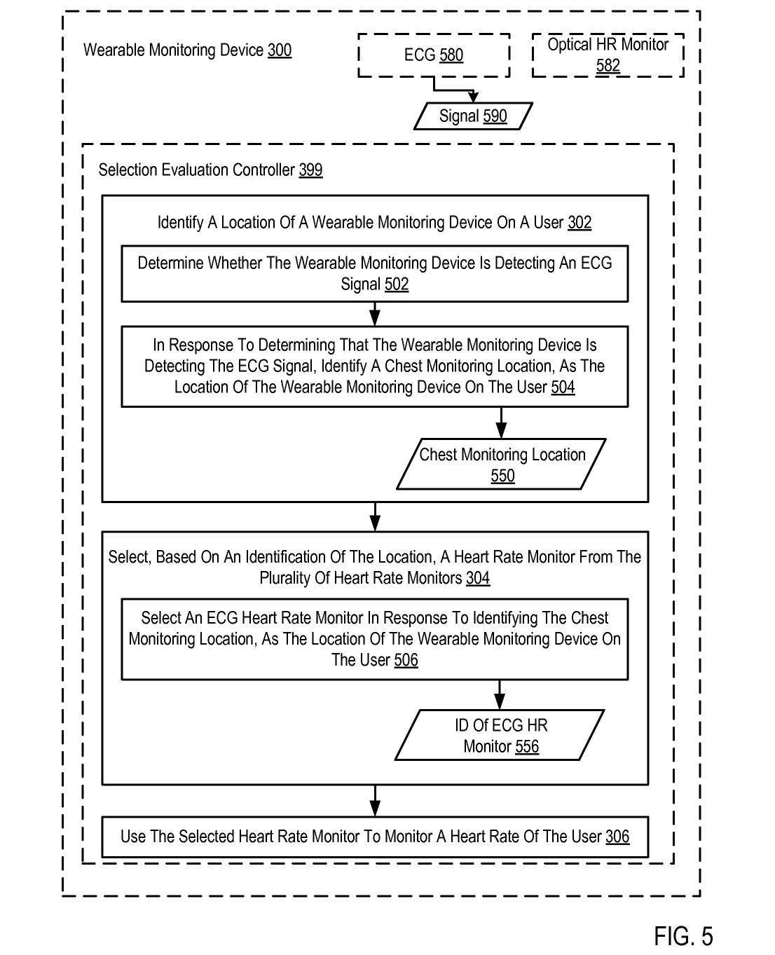

For further explanation, FIG. 5 sets forth a flow chart illustrating another example embodiment of a method for choosing a heart rate monitor for a wearable monitoring device. In the example of FIG. 5, an ECG heart rate monitor (580) and an optical heart rate monitor (582) are included in the wearable monitoring device (300). The method of FIG. 5 is similar to the method of FIG. 3 in that the method of FIG. 5 also includes a selection evaluation controller (399) identifying (302) a location of the wearable monitoring device (300) on a user (380); selecting (304), based on an identification (350) of the location, a heart rate monitor (354) from a plurality of heart rate monitors (320); and using (306) the selected heart rate monitor (354) to monitor a heart rate of the user (380).

In the method of FIG. 5, however, identifying (302) a location of the wearable monitoring device (300) on a user (380) includes determining (502) whether the wearable monitoring device (300) is detecting an ECG signal (590). Determining (502) whether the wearable monitoring device (300) is detecting an ECG signal (590) may be carried out by receiving output from the ECG heart rate monitor (580); polling or querying the ECG heart rate monitor (580); and receiving an indication from the ECG heart rate monitor (580) indicating that the ECG heart rate monitor is detecting the ECG signal (590).

In the method of FIG. 5, however, identifying (302) a location of the wearable monitoring device (300) on a user (380) includes in response to determining that the wearable monitoring device (300) is detecting the ECG signal (590), identifying (504) a chest monitoring location (550), as the location of the wearable monitoring device (300 on the user (380). Identifying (504) a chest monitoring location (550), as the location of the wearable monitoring device (300 on the user (380) may be carried out by examining a table containing an entry that matches the detection of an ECG signal with a chest monitoring location. For example, if the heart rate monitor (580) is detecting an activity pattern based on the electrical activity of the heart, then the heart rate monitor may be positioned near the user's heart in the chest monitoring location.

In the method of FIG. 5, selecting (304), based on the identified location, a heart rate monitor (354) from a plurality of heart rate monitors (320) includes selecting (506) an ECG heart rate monitor (580) from the plurality of heart rate monitors in response to identifying the chest monitoring location (550), as the location of the wearable monitoring device (300) on the user (380). Selecting (506) an ECG heart rate monitor (580) from the plurality of heart rate monitors in response to identifying the chest monitoring location (550), as the location of the wearable monitoring device (300) on the user (380) may be carried out by examining a table containing an entry that matches the chest monitoring location with an ECG heart rate monitor.

In the example of FIG. 5, the selection evaluation controller selects the ECG heart rate monitor if the ECG heart rate monitor is detecting an activity pattern based on an electrical activity of the user's heart. In a particular embodiment, the ECG heart rate monitor uses less energy and is more efficient than an optical heart rate monitor. In that embodiment, the selection evaluation controller may select the ECG heart rate monitor any time the selection evaluation controller determines that the ECG heart rate monitor is available as indicated by the wearable monitoring device being in the chest monitoring position.

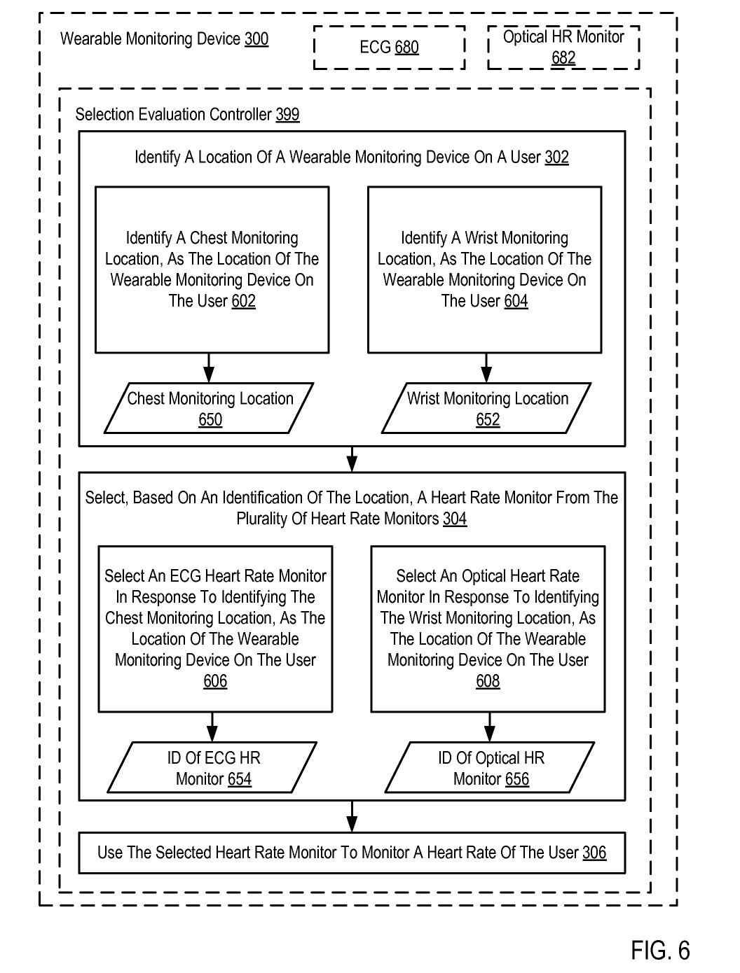

For further explanation, FIG. 6 sets forth a flow chart illustrating another example embodiment of a method for choosing a heart rate monitor for a wearable monitoring device. The method of FIG. 6 is similar to the method of FIG. 3 in that the method of FIG. 6 also includes a selection evaluation controller (399) identifying (302) a location of the wearable monitoring device (300) on a user (380); selecting (304), based on an identification (350) of the location, a heart rate monitor (354) from a plurality of heart rate monitors (320); and using (306) the selected heart rate monitor (354) to monitor a heart rate of the user (380).

In the method of FIG. 6, however, identifying (302) a location of the wearable monitoring device (300) on a user (380) optionally includes identifying (602) a chest monitoring location (650), as the location of the wearable monitoring device (300) on the user (380). Identifying (602) a chest monitoring location (650), as the location of the wearable monitoring device (300) on the user (380) may be carried out by receiving user input directly or indirectly indicating that the wearable monitoring device is located on the chest monitoring location. Identifying (602) a chest monitoring location (650), as the location of the wearable monitoring device (300) on the user (380) may also be carried out by analyzing sensor input to determine that the wearable monitoring device is located on the chest monitoring location.

In the method of FIG. 6, identifying (302) a location of the wearable monitoring device (300) on a user (380) optionally includes identifying (604) a wrist monitoring location (652), as the location of the wearable monitoring device (300) on the user (380). Identifying (604) a wrist monitoring location (652), as the location of the wearable monitoring device (300) on the user (380) may be carried out by receiving user input directly or indirectly indicating that the wearable monitoring device is located on the wrist monitoring location. Identifying (604) a wrist monitoring location (652), as the location of the wearable monitoring device (300) on the user (380) may be carried out by analyzing sensor input to determine that the wearable monitoring device is located on the wrist monitoring location.

In the method of FIG. 6, selecting (304), based on the identified location, a heart rate monitor (354) from a plurality of heart rate monitors (320) optionally includes selecting (606) an ECG heart rate monitor (680) in response to identifying the chest monitoring location (650), as the location of the wearable monitoring device (300) on the user (380). Selecting (606) an ECG heart rate monitor (680) in response to identifying the chest monitoring location (650), as the location of the wearable monitoring device (300) on the user (380) may be carried out by examining a table containing entries that match the chest monitoring location with the ECG heart rate monitor.

In the method of FIG. 6, selecting (304), based on the identified location, a heart rate monitor (354) from a plurality of heart rate monitors (320) optionally includes selecting (608) an optical heart rate monitor (682) in response to identifying the wrist monitoring location (652), as the location of the wearable monitoring device (300) on the user (380). Selecting (608) an optical heart rate monitor (682) in response to identifying the wrist monitoring location (652), as the location of the wearable monitoring device (300) on the user (380) may be carried out by examining a table containing entries that match the wrist monitoring location with the optical heart rate monitor.

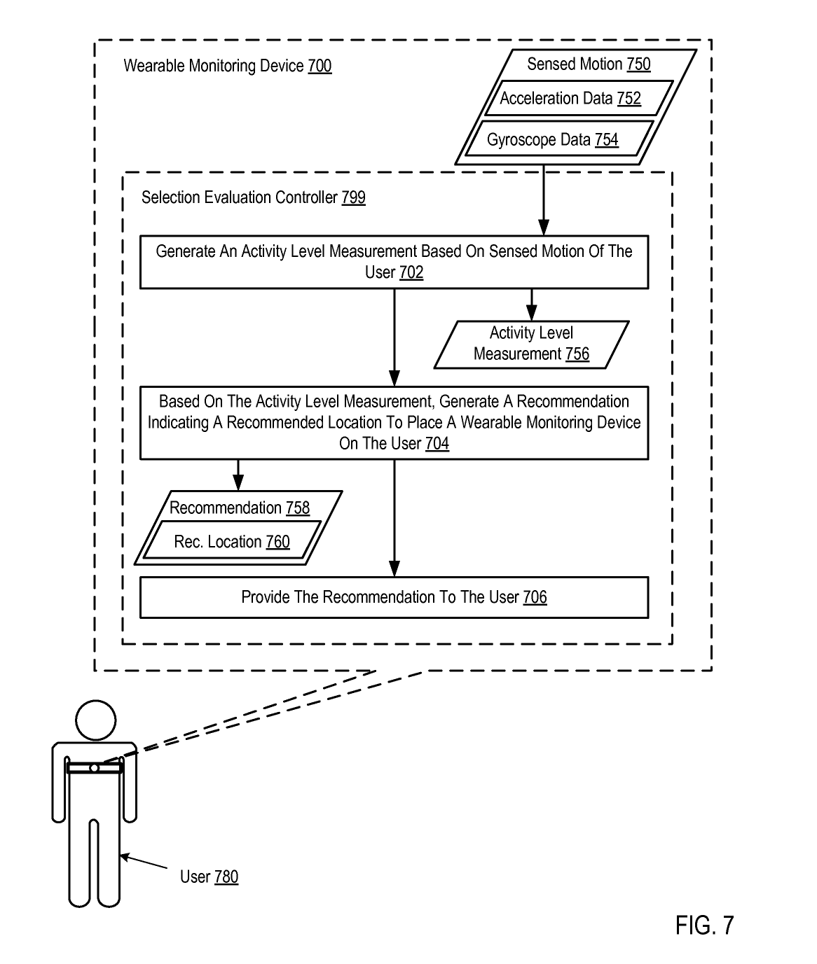

For further explanation, FIG. 7 sets forth a flow chart illustrating another example embodiment of a method for choosing a heart rate monitor for a wearable monitoring device. The method of FIG. 7 includes a selection evaluation controller (799) of a wearable monitoring device (700) generating (702) an activity level measurement (756) based on sensed motion (750) of the user (780). The sensed motion of the user may be indicated by motion data generated by one or more motion sensors of the wearable monitoring device. Non-limiting examples of motion data include acceleration data and gyroscope data, generated by an accelerometer and a gyroscope, respectively. Generating (702) an activity level measurement (756) based on sensed motion (750) of the user (780) may be carried out by determining an activity level measurement that indicates an intensity level of the activity of the user. For example, an activity level measurement may indicate an absolute value of acceleration data indicating changes in the direction of motion by the user.

The method of FIG. 7 also includes the selection evaluation controller (799) based on the activity level measurement (756), generating (704) a recommendation (758) indicating a recommended location (760) to place the wearable monitoring device (700) on the user (780). Generating (704) a recommendation (758) indicating a recommended location (760) to place the wearable monitoring device (700) on the user (780) may be carried out by determining which heart rate sensor best corresponds to the intensity level of the user's exercise activity; and storing an indication of the recommended location within a visual output message or an audio output message. For example, a selection evaluation controller may determine that an ECG heart rate monitor is more accurate at higher levels of exercise activity than an optical heart rate monitor. In this example, the selection evaluation monitor (199) may recommend that a user move the wearable monitoring device to the chest monitoring location so that the ECG heart rate monitor may be used.

The method of FIG. 7 also includes the selection evaluation controller (799) providing (706) the recommendation (758) to the user (780). Providing (706) the recommendation (758) to the user (780) may be carried out by sending a recommendation message to the user suggesting a position for the wearable monitoring device. In a particular embodiment, the recommendation message is a visual output message or an audio output message.

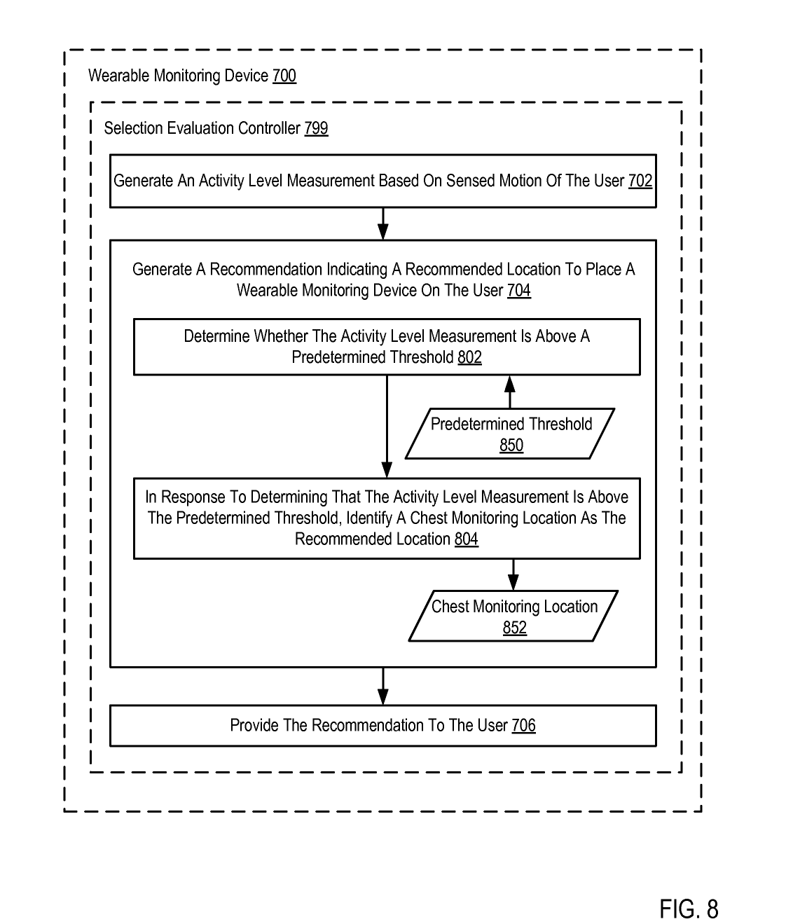

For further explanation, FIG. 8 sets forth a flow chart illustrating another example embodiment of a method for choosing a heart rate monitor for a wearable monitoring device. The method of FIG. 8 is similar to the method of FIG. 7 in that the method of FIG. 8 also includes a selection evaluation controller (799) generating (702) an activity level measurement (756) based on sensed motion (750) of the user (780); based on the activity level measurement (756), generating (704) a recommendation (758) indicating a recommended location (760) to place the wearable monitoring device (700) on the user (780); and providing (706) the recommendation (758) to the user (780).

In the method of FIG. 8, however, generating (704) a recommendation (758) indicating a recommended location (760) to place the wearable monitoring device (700) on the user (780) includes determining (802) whether the activity level measurement (756) is above a predetermined threshold (850). Determining (802) whether the activity level measurement (756) is above a predetermined threshold (850) may be carried out by comparing the activity level measurement to the predetermined threshold. In a particular embodiment, the selection evaluation controller (799) identifies a predetermined threshold from a plurality of predetermined thresholds based on the type and position of the sensor that generated the motion data. For example, an activity level measurement that is based on acceleration data may have a corresponding first predetermined threshold. As another example, an activity level measurement that is based on gyroscope data may have a corresponding second predetermined threshold.

In the method of FIG. 8, generating (704) a recommendation (758) indicating a recommended location (760) to place the wearable monitoring device (700) on the user (780) also includes in response to determining that the activity level measurement (756) is above the predetermined threshold (850), identifying (804) a chest monitoring location (852) as the recommended location (760). Identifying (804) a chest monitoring location (852) as the recommended location (760) may be carried out by analyzing sensor input to determine that the wearable monitoring device is located on the chest monitoring location.

For example, if the user is wearing the wearable monitoring device in the wrist monitoring location, the wearable monitoring device may utilize an optical heart monitor to determine the heart rate of the user. Continuing with this example, if the user begins running, the selection evaluation controller (799) may determine that the user's activity level measurement is higher than an acceptable level and in response, suggest that the user switch the wearable monitoring device to the chest monitoring position. In this example, by switching to the chest monitoring position, the wearable monitoring device may be able to select and use the ECG heart rate monitor instead of the optical heart rate monitor. As explained above, in a particular embodiment, the ECG heart rate monitor is more power efficient and more accurate than the optical heart rate monitor. Therefore, the selection evaluation controller may indicate the chest monitoring location as the recommended location so that the wearable monitoring device can utilize the more efficient and accurate ECG heart rate monitor.

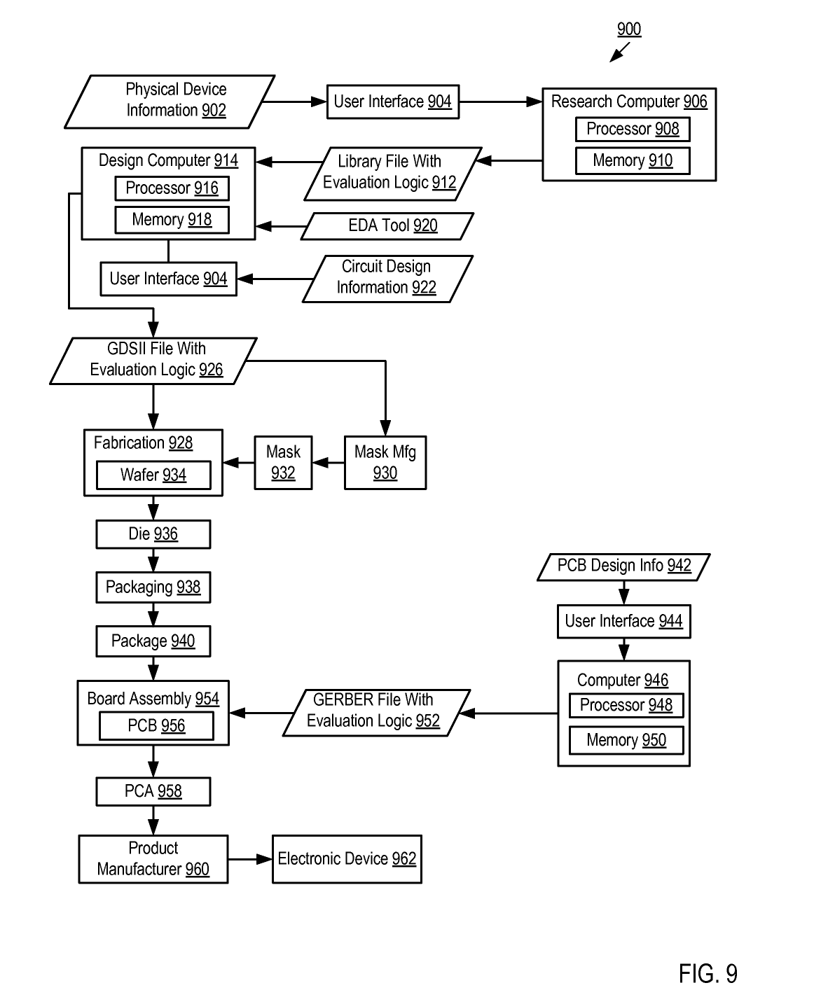

For further explanation, FIG. 9 sets forth a data flow diagram illustrating a manufacturing process (900) for a device that includes a selection evaluation controller. Physical device information (902 is received at the manufacturing process (900), such as at a research computer (906). The physical device information (902) may include design information representing at least one physical property of a semiconductor device, such as a device that includes the selection evaluation controller (199) of FIG. 1 (e.g., the wearable monitoring devices (101, 111) of FIG. 1 and the wearable monitoring device (200) of FIG. 2). For example, the physical device information (902) may include physical parameters, material characteristics, and structure information that is entered via a user interface (904) coupled to the research computer (906). The research computer (906) includes a processor (908), such as one or more processing cores, coupled to a computer readable medium such as a memory (910). The memory (910) may store computer readable instructions that are executable to cause the processor (908) to transform the physical device information (902) to comply with a file format and to generate a library file (912) containing evaluation logic for choosing a heart rate monitor for a wearable monitoring device.

In a particular embodiment, the library file (912) includes at least one data file including the transformed design information. For example, the library file (912) may include a library of semiconductor devices including a device that includes the selection evaluation controller (199) of FIG. 1 (e.g., the wearable monitoring devices (101, 111) of FIG. 1 and the wearable monitoring device (200) of FIG. 2) that is provided to use with an electronic design automation (EDA) tool (920).

The library file (912) may be used in conjunction with the EDA tool (920) at a design computer (914) including a processor (916), such as one or more processing cores, coupled to a memory (918). The EDA tool (920) may be stored as processor executable instructions at the memory (918) to enable a user of the design computer (914) to design a circuit including a device that includes the selection evaluation controller (199) of FIG. 1 (e.g., the wearable monitoring devices (101, 111) of FIG. 1 and the wearable monitoring device (200) of FIG. 2) of the library file (912). For example, a user of the design computer (914) may enter circuit design information (922) via a user interface (924) coupled to the design computer (914). The circuit design information (922) may include design information representing at least one physical property of a semiconductor device, such as a device that includes the selection evaluation controller (199) of FIG. 1 (e.g., the wearable monitoring devices (101, 111) of FIG. 1 and the wearable monitoring device (200) of FIG. 2). To illustrate, the circuit design property may include identification of particular circuits and relationships to other elements in a circuit design, positioning information, feature size information, interconnection information, or other information representing a physical property of a semiconductor device.

The design computer (914) may be configured to transform the design information, including the circuit design information (922), to comply with a file format. To illustrate, the file formation may include a database binary file format representing planar geometric shapes, text labels, and other information about a circuit layout in a hierarchical format, such as a Graphic Data System (GDSII) file format. The design computer (914) may be configured to generate a data file including the transformed design information, such as a GDSII file (926) that includes information describing a device that includes evaluation logic used by the selection evaluation controller (199) of FIG. 1 (e.g., the wearable monitoring devices (101, 111) of FIG. 1 and the wearable monitoring device (200) of FIG. 2) for choosing a heart rate monitor for a wearable monitoring device in addition to other circuits or information. To illustrate, the data file may include information corresponding to a system-on-chip (SOC) that includes a device that includes the selection evaluation controller (199) of FIG. 1 (e.g., the wearable monitoring devices (101, 111) of FIG. 1 and the wearable monitoring device (200) of FIG. 2) and that also includes additional electronic circuits and components within the SOC.

The GDSII file (926) may be received at a fabrication process (928) to manufacture a device that includes the selection evaluation controller (199) of FIG. 1 (e.g., the wearable monitoring devices (101, 111) of FIG. 1 and the wearable monitoring device (200) of FIG. 2) according to transformed information in the GDSII file (926). For example, a device manufacture process may include providing the GDSII file (926) to a mask manufacturer (930) to create one or more masks, such as masks to be used with photolithography processing, illustrated as a representative mask (932). The mask (932) may be used during the fabrication process to generate one or more wafers (934), which may be tested and separated into dies, such as a representative die (936). The die (936) includes a circuit including a device that includes the selection evaluation controller (199) of FIG. 1 (e.g., the wearable monitoring devices (101, 111) of FIG. 1 and the wearable monitoring device (200) of FIG. 2).

The die (936) may be provided to a packaging process (938) where the die (936) is incorporated into a representative package (940). For example, the package (940) may include the single die (936) or multiple dies, such as a system-in-package (SiP) arrangement. The package (940) may be configured to conform to one or more standards or specifications, such as Joint Electron Device Engineering Council (JEDEC) standards.

Information regarding the package (940) may be distributed to various product designers, such as via a component library stored at a computer (946). The computer (946) may include a processor (948), such as one or more processing cores, coupled to a memory (950). A printed circuit board (PCB) tool may be stored as processor executable instructions at the memory (950) to process PCB design information (942) received from a user of the computer (946) via a user interface (944). The PCB design information (942) may include physical positioning information of a packaged semiconductor device on a circuit board, the packaged semiconductor device corresponding to the package (940) including a device that includes the selection evaluation controller (199) of FIG. 1 (e.g., the wearable monitoring devices (101, 111) of FIG. 1 and the wearable monitoring device (200) of FIG. 2).

The computer (946) may be configured to transform the PCB design information (942) to generate a data file, such as a GERBER file (952) with data that includes physical positioning information of a packaged semiconductor device on a circuit board, as well as layout of electrical connections such as traces and vias, where the packaged semiconductor device corresponds to the package (940) including the selection evaluation controller (199) of FIG. 1. In other embodiments, the data file generated by the transformed PCB design information may have a format other than a GERBER format.

The GERBER file (952) may be received at a board assembly process (954) and used to create PCBs, such as a representative PCB (956), manufactured in accordance with the design information stored within the GERBER file (952). For example, the GERBER file (952) may be uploaded to one or more machines to perform various steps of a PCB production process. The PCB (956) may be populated with electronic components including the package (940) to form a representative printed circuit assembly (PCA) (958).

The PCA (958) may be received at a product manufacture process (960) and integrated into one or more electronic devices, such as a first representative electronic device (962) and a second representative electronic device (964). As an illustrative, non-limiting example, the first representative electronic device (962), the second representative electronic device (964), or both, may be selected from the group of a set top box, a music player, a video player, an entertainment unit, a navigation device, a communications device, a personal digital assistant (PDA), a fixed location data unit, and a computer, into which the at least one controllable energy consuming module is integrated. As another illustrative, non-limiting example, one or more of the electronic devices (962) and (964) may be remote units such as mobile phones, hand-held personal communication systems (PCS) units, portable data units such as personal data assistants, global positioning system (GPS) enabled devices, navigation devices, fixed location data units such as meter reading equipment, or any other device that stores or retrieves data or computer instructions, or any combination thereof. Although FIG. 8 illustrates remote units according to teachings of the disclosure, the disclosure is not limited to these exemplary illustrated units. Embodiments of the disclosure may be suitably employed in any device which includes active integrated circuitry including memory and on-chip circuitry.

A device that includes the selection evaluation controller (199) of FIG. 1 may be fabricated, processed, and incorporated into an electronic device, as described in the illustrative process (900). One or more aspects of the embodiments disclosed with respect to FIGS. 1-8 may be included at various processing stages, such as within the library file (912), the GDSII file (926), and the GERBER file (952), as well as stored at the memory (910) of the research computer (906), the memory (918) of the design computer (914), the memory (950) of the computer (946), the memory of one or more other computers or processors (not shown) used at the various stages, such as at the board assembly process (954), and also incorporated into one or more other physical embodiments such as the mask (932), the die (936), the package (940), the PCA (958), other products such as prototype circuits or devices (not shown), or any combination thereof. For example, the GDSII file (926) or the fabrication process (928) can include a computer readable tangible medium storing instructions executable by a computer, the instructions including instructions that are executed by the computer to perform the methods of FIGS. 3-8, or any combination thereof. Although various representative stages of production from a physical device design to a final product are depicted, in other embodiments fewer stages may be used or additional stages may be included. Similarly, the process (900) may be performed by a single entity, or by one or more entities performing various stages of the process (900).

Those of skill would further appreciate that the various illustrative logical blocks, configurations, modules, circuits, and method steps described in connection with the embodiments disclosed herein may be implemented as electronic hardware, computer software executed by a processing unit, or combinations of both. Various illustrative components, blocks, configurations, modules, circuits, and steps have been described above generally in terms of their functionality. Whether such functionality is implemented as hardware or executable processing instructions depends on the particular application and design constraints imposed on the overall system. Skilled artisans may implement the described functionality in varying ways with each particular application, but such implementation decisions should not be interpreted as causing a departure from the scope of the present disclosure.

The steps of a method or algorithm described in connection with the embodiments disclosed herein may be embodied directly in hardware, in a software module executed by a processor, or in a combination of the two. A software module may reside in random access memory (RAM), a magnetoresistive random access memory (MRAM), a spin-torque-transfer MRAM (STT-MRAM), flash memory, read-only memory (ROM), programmable read-only memory (PROM), erasable programmable read-only memory (EPROM), electrically erasable programmable read-only memory (EEPROM), registers, hard disk, a removable disk, a compact disc read-only memory (CD-ROM), or any other form of storage medium known in the art. An exemplary storage medium is coupled to the processor such that the processor can read information from, and write information to, the storage medium. In the alternative, the storage medium may be integral to the processor. The processor and the storage medium may reside in an application-specific integrated circuit (ASIC). The ASIC may reside in a computing device or a user terminal. In the alternative, the processor and the storage medium may reside as discrete components in a computing device or user terminal.

The previous description of the disclosed embodiments is provided to enable any person skilled in the art to make or use the disclosed embodiments. Various modifications to these embodiments will be readily apparent to those skilled in the art, and the principles defined herein may be applied to other embodiments without departing from the scope of the disclosure. Thus, the present disclosure is not intended to be limited to the embodiments shown herein but is to be accorded the widest scope possible consistent with the principles and novel features as defined by the following claims.

What is claimed is:

1. A method for choosing a heart rate monitor for a wearable monitoring device, the method comprising:
identifying, by a selection evaluation controller, a location of a wearable monitoring device on a user; wherein the wearable monitoring device includes a plurality of heart rate monitors;
selecting based on an identification of the location, by the selection evaluation controller, a heart rate monitor from the plurality of heart rate monitors; and
using, by the selection evaluation controller, the selected heart rate monitor to monitor a heart rate of the user.

2. The method of claim 1 wherein identifying a location of a wearable monitoring device on a user includes:
determining whether the wearable monitoring device is coupled to a docking device;
in response to determining that the wearable monitoring device is coupled to the docking device, determining a type of the docking device; and
identifying the location of the wearable monitoring device based on the determined type of the docking device.

3. The method of claim 1 wherein identifying a location of a wearable monitoring device on a user includes:
determining whether the wearable monitoring device is detecting an ECG signal;
in response to determining that the wearable monitoring device is detecting the ECG signal, identifying a chest monitoring location, as the location of the wearable monitoring device on the user;
wherein selecting based on an identification of the location, by the selection evaluation controller, a heart rate monitor from the plurality of heart rate monitors includes selecting an ECG heart rate monitor from the plurality of heart rate monitors in response to identifying the chest monitoring location, as the location of the wearable monitoring device on the user.

4. The method of claim 1 wherein each heart rate monitor is a different type of heart rate monitor.

5. The method of claim 1 wherein the plurality of heart rate monitors includes an ECG heart rate monitor and an optical heart rate monitor.

6. The method of claim 1 wherein selecting based on an identification of the location, by the selection evaluation controller, a heart rate monitor from the plurality of heart rate monitors includes selecting an ECG heart rate monitor in response to identifying the chest monitoring location, as the location of the wearable monitoring device on the user.

7. The method of claim 1 wherein selecting based on an identification of the location, by the selection evaluation controller, a heart rate monitor from the plurality of heart rate monitors includes selecting an optical heart rate monitor in response to identifying the wrist monitoring location, as the location of the wearable monitoring device on the user.

8. A method for choosing a heart rate monitor for a wearable monitoring device, the method comprising:
generating, by a selection evaluation controller, an activity level measurement based on sensed motion of the user;
based on the activity level measurement, generating, by the selection evaluation controller, a recommendation indicating a recommended location to place a wearable monitoring device on the user; and
providing, by the selection evaluation controller, the recommendation to the user.

9. The method of claim 8 wherein the sensed motion of the user includes at least one of acceleration data and gyroscope data.

10. The method of claim 8 wherein generating a recommendation indicating a recommended location to place a wearable monitoring device on the user includes:
determining whether the activity level measurement is above a predetermined threshold; and
in response to determining that the activity level measurement is above the predetermined threshold, identifying a chest monitoring location as the recommended location.

11. An apparatus for choosing a heart rate monitor for a wearable monitoring device, the apparatus comprising a computer processor and computer memory operatively coupled to the computer processor, the computer memory having disposed within it computer program instructions that, when executed by the computer processor, cause the apparatus to carry out the steps of:
identifying, by a selection evaluation controller, a location of a wearable monitoring device on a user; wherein the wearable monitoring device includes a plurality of heart rate monitors;
selecting based on an identification of the location, by the selection evaluation controller, a heart rate monitor from the plurality of heart rate monitors; and
using, by the selection evaluation controller, the selected heart rate monitor to monitor a heart rate of the user.

12. The apparatus of claim 11 wherein identifying a location of a wearable monitoring device on a user includes:
determining whether the wearable monitoring device is coupled to a docking device;
in response to determining that the wearable monitoring device is coupled to the docking device, determining a type of the docking device; and
identifying the location of the wearable monitoring device based on the determined type of the docking device.

13. The apparatus of claim 11 wherein identifying a location of a wearable monitoring device on a user includes:
determining whether the wearable monitoring device is detecting an ECG signal;
in response to determining that the wearable monitoring device is detecting the ECG signal, identifying a chest monitoring location, as the location of the wearable monitoring device on the user;
wherein selecting based on an identification of the location, by the selection evaluation controller, a heart rate monitor from the plurality of heart rate monitors includes selecting an ECG heart rate monitor from the plurality of heart rate monitors in response to identifying the chest monitoring location, as the location of the wearable monitoring device on the user.

14. The apparatus of claim 11 wherein each heart rate monitor is a different type of heart rate monitor.

15. The apparatus of claim 11 wherein the plurality of heart rate monitors includes an ECG heart rate monitor and an optical heart rate monitor.

16. The apparatus of claim 11 wherein selecting based on an identification of the location, by the selection evaluation controller, a heart rate monitor from the plurality of heart rate monitors includes selecting an ECG heart rate monitor in response to identifying the chest monitoring location, as the location of the wearable monitoring device on the user.

17. The apparatus of claim 11 wherein selecting based on an identification of the location, by the selection evaluation controller, a heart rate monitor from the plurality of heart rate monitors includes selecting an optical heart rate monitor in response to identifying the wrist monitoring location, as the location of the wearable monitoring device on the user.

18. A computer readable storage medium storing instructions executable by a computer for choosing a heart rate monitor for a wearable monitoring device, the instructions comprising:
instructions that are executable by the computer to identify, by a selection evaluation controller, a location of a wearable monitoring device on a user; wherein the wearable monitoring device includes a plurality of heart rate monitors;
instructions that are executable by the computer to select based on an identification of the location, by the selection evaluation controller, a heart rate monitor from the plurality of heart rate monitors; and
instructions that are executable by the computer to use, by the selection evaluation controller, the selected heart rate monitor to monitor a heart rate of the user.

19. The computer readable storage medium of claim 18 wherein the instructions are executable by a processor integrated into a device selected from the group consisting of a navigation device, a communications device, a personal digital assistant (PDA), a fixed location data unit, and a computer.
